(19) United States
(12) Patent Application Publication
(10) Pub. No.: US 2024/0293016 A1
(43) Pub. Date: Sep. 5, 2024

(54) IMAGE PROCESSING APPARATUS, FLUORESCENCE-IMAGE PROCESSING METHOD, AND COMPUTER-READABLE RECORDING MEDIUM

(71) Applicant: OLYMPUS CORPORATION, Tokyo (JP)

(72) Inventor: Kazuaki WAKANA, Tokyo (JP)

(73) Assignee: OLYMPUS CORPORATION, Tokyo (JP)

(21) Appl. No.: 18/661,819

(22) Filed: May 13, 2024

Related U.S. Application Data
(63) Continuation of application No. PCT/JP2021/048668, filed on Dec. 27, 2021.

(51) Int. Cl. A61B 1/04; A61B 1/00

(52) U.S. Cl. CPC A61B 1/043; A61B 1/000095

(57) ABSTRACT

Provided is an image processing apparatus that processes a fluorescence image signal. The image processing apparatus includes a processor including hardware, the processor being configured to: acquire a fluorescence image obtained by therapeutic light that causes a drug to react; set a fluorescence intensity signal in which an occurrence frequency of a fluorescence intensity of the fluorescence image is equal to or higher than a threshold to a tone adjustment range of an image; and allocate tones according to the set tone adjustment range to generate a tone-expanded image.

### CROSS-REFERENCE TO RELATED APPLICATION

This application is a continuation of International Application No. PCT/JP2021/048668, filed on Dec. 27, 2021, the entire contents of which are incorporated herein by reference.

### BACKGROUND

### 1. Technical Field

The present disclosure relates to an image processing apparatus, a fluorescence-image processing method, and a computer-readable recording medium.

### 2. Related Art

In the related art, research is in progress on photoimmunotherapy (PIT) for treating cancers by specifically binding an antibody drug to cancer cell proteins, and by activating the antibody drug with as near-infrared light, which is therapeutic light, to destroy cancer cells. The antibody drug irradiated with near-infrared light causes cancer cells to swell and induce cell death of the cancer cells. In this process, the antibody drug is excited and thereby emit fluorescence. The intensity of this fluorescence is used as an indicator of treatment effectiveness.

Additionally, as a technique for evaluating treatment based on the intensity of fluorescence, a technique of observing subcutaneous blood circulation by using indocyanine green (ICG) introduced into the bloodstream and imaging the fluorescence of this ICG has been known (for example, JP-A-2016-135253). In JP-A-2016-135253, contrast adjustment and dynamic range compression are performed to represent overall tones in observation images.

### SUMMARY

In some embodiments, Provided is an image processing apparatus that processes a fluorescence image signal. The image processing apparatus includes a processor including hardware, the processor being configured to: acquire a fluorescence image obtained by therapeutic light that causes a drug to react; set a fluorescence intensity signal in which an occurrence frequency of a fluorescence intensity of the fluorescence image is equal to or higher than a threshold to a tone adjustment range of an image; and allocate tones according to the set tone adjustment range to generate a tone-expanded image.

In some embodiments, a fluorescence-image processing method includes: acquiring, by a processor, a fluorescence image obtained by therapeutic light that causes a drug to react; setting, by the processor, a fluorescence intensity signal in which an occurrence frequency of a fluorescence intensity of the fluorescence image is equal to or higher than a threshold to a tone adjustment range of an image; and allocating, by the processor, tones according to the set tone adjustment range to generate a tone-expanded image.

In some embodiments, provided is a non-transitory computer-readable recording medium with an executable image processing program stored thereon. The program causes a computer to execute: acquiring a fluorescence image obtained by therapeutic light that causes a drug to react; setting a fluorescence intensity signal in which an occurrence frequency of a fluorescence intensity of the fluorescence image is equal to or higher than a threshold to a tone adjustment range of an image; and allocating tones according to the tone adjustment range set at the setting to generate a tone-expanded image.

In some embodiments, provided is an image processing apparatus that processes a fluorescence image signal. The image processing apparatus includes: a processor including hardware, the processor being configured to: acquire an initial fluorescence image at a time of starting irradiation of therapeutic light that causes a drug to react; acquire a fluorescence image during irradiation of the therapeutic light; generate a difference image that represents a difference in a fluorescence intensity between the initial fluorescence image and the fluorescence image during irradiation of the therapeutic light; set a tone range of an image with respect to the difference based on a distribution of a fluorescence intensity of the fluorescence image; and allocate tones according to the set tone range to generate a tone-expanded image.

In some embodiments, provided is an image processing apparatus that processes a fluorescence image. The image processing apparatus includes: a processor including hardware, the processor being configured to: acquire a fluorescence image obtained by therapeutic light that causes a drug to react; set a tone range of an image based on a distribution of a fluorescence intensity of the fluorescence image and on an attenuation target value set with respect to the fluorescence image; and allocate tones according to the set tone range to generate a tone-expanded image.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to a first embodiment of the disclosure;

FIG. 2 is a block diagram illustrating a schematic configuration of the endoscope system according to the first embodiment of the disclosure;

FIG. 3 is a diagram explaining a distal end configuration of an endoscope according to the first embodiment of the disclosure;

FIG. 4 is a diagram illustrating an example of a flow of treatment using the endoscope according to the first embodiment of the disclosure;

FIG. 5 is a flowchart illustrating an example of processing of a processing apparatus according to the first embodiment of the disclosure;

FIG. 6 is a diagram illustrating an example of an observation image including a fluorescence image;

FIG. 7 is a diagram illustrating a fluorescence image indicating a fluorescence intensity in the observation image illustrated in FIG. 6;

FIG. 8 is a diagram illustrating a relationship between the fluorescence intensity and an occurrence frequency of in a region of interest;

FIG. 9 is a diagram (Part 1) illustrating an example of a fluorescence intensity distribution with respect to pixels of a fluorescence image;

FIG. 10 is a diagram (Part 2) illustrating an example of the fluorescence intensity distribution with respect to pixels in the fluorescence image;

FIG. 11 is a diagram illustrating an example of a tone-expanded image obtained by applying tone enhancement processing to the fluorescence image illustrated in FIG. 7;

FIG. 12 is a diagram (Part 1) illustrating another display example of the tone-expanded image obtained by applying the tone enhancement processing to the fluorescence image illustrated in FIG. 7;

FIG. 13 is a diagram (Part 2) illustrating another display example of the tone-expanded image obtained by applying the tone enhancement processing to the fluorescence image illustrated in FIG. 7;

FIG. 14 is a diagram (Part 1) for explaining about the tone enhancement processing according to the first embodiment of the disclosure;

FIG. 15 is a diagram for explaining about clipping processing according to a modification of the first embodiment of the disclosure;

FIG. 16 is a block diagram illustrating a schematic configuration of an endoscope system according to a second embodiment of the disclosure;

FIG. 17 is a flowchart illustrating an example of processing of a processing apparatus according to the second embodiment of the disclosure;

FIG. 18 is a diagram illustrating an example of fluorescence intensities of corresponding pixels acquired at different times;

FIG. 19 is a diagram illustrating an example of a difference image;

FIG. 20 is a diagram (Part 1) illustrating an example of a distribution of fluorescence intensity difference for pixels in a difference image and an example of a tone range;

FIG. 21 is a diagram (Part 2) illustrating an example of a distribution of fluorescence intensity difference for pixels in a difference image and an example of a tone range;

FIG. 22 is a diagram illustrating an example of a tone-expanded image obtained by applying tone enhancement processing to the difference image illustrated in FIG. 19;

FIG. 23 is a diagram illustrating an example of fluorescence intensities of corresponding pixels acquired at different times;

FIG. 24 is a diagram illustrating an example of a difference image;

FIG. 25 is a diagram (Part 1) illustrating an example of a distribution of fluorescence intensity difference for pixels in a difference image and an example of a tone range;

FIG. 26 is a diagram (Part 2) illustrating an example of a distribution of fluorescence intensity difference for pixels in a difference image and an example of a tone range;

FIG. 27 is a diagram illustrating an example of a tone-expanded image obtained by applying tone enhancement processing to the difference image illustrated in FIG. 24;

FIG. 28 is a flowchart illustrating an example of processing of a processing apparatus according to a third embodiment of the disclosure;

FIG. 29 is a diagram illustrating an example of fluorescence intensities of corresponding pixels acquired at different times;

FIG. 30 is a diagram (Part 1) illustrating an example of a distribution of fluorescence intensity difference for pixels in a fluorescence image;

FIG. 31 is a diagram (Part 2) illustrating an example of a distribution of fluorescence intensity difference for pixels in a fluorescence image; and

FIG. 32 is a diagram explaining about an example of setting of a minimum value of the tone range.

### DETAILED DESCRIPTION

Hereinafter, modes (hereinafter, “embodiments”) to implement the disclosure will be explained. In the embodiments, as an example of a system including an image processing apparatus, a photoimmunotherapy system, and a fluorescence endoscope according to the disclosure, a medical endoscope system that captures an image of an inside of a body of a subject, such as a patient, and displays it will be explained. Moreover, the embodiments are not intended to limit the disclosure. Furthermore, identical reference symbols are assigned to identical components in description of the drawings to be explained.

### First Embodiment

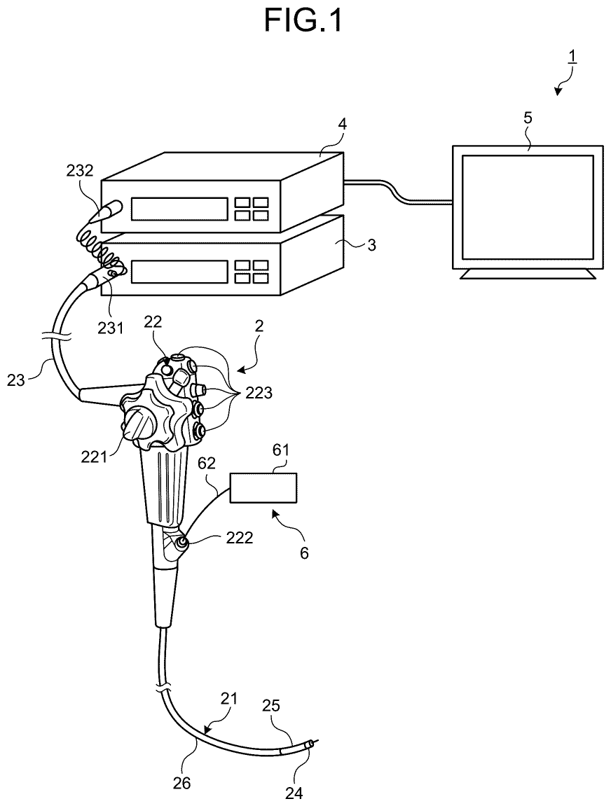

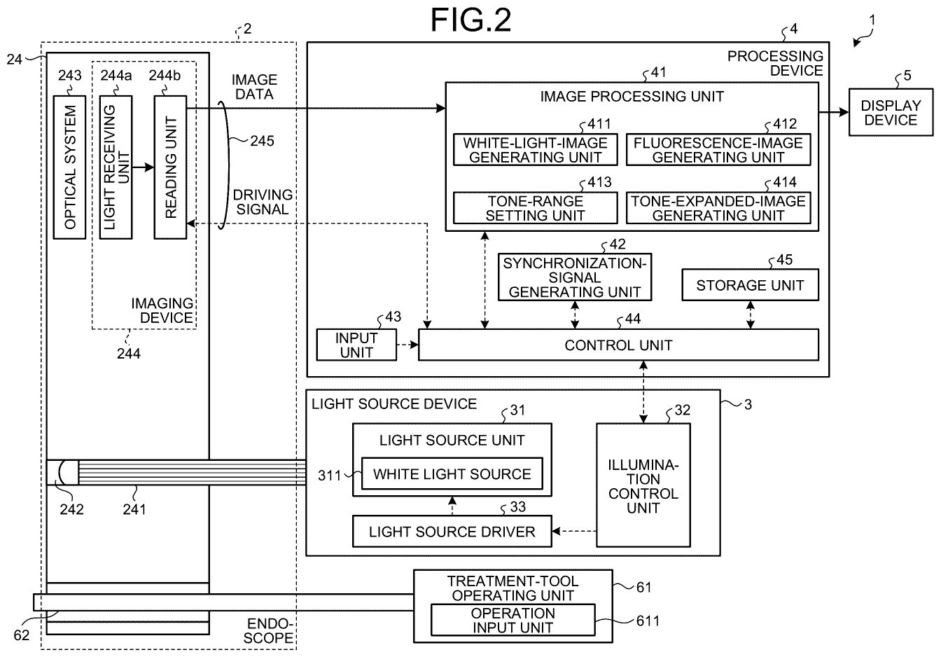

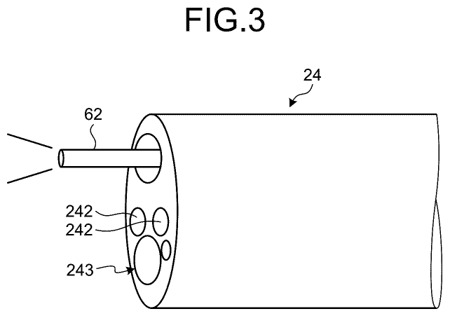

FIG. 1 is a diagram illustrating a schematic configuration of an endoscope system according to a first embodiment of the disclosure. FIG. 2 is a block diagram illustrating a schematic configuration of the endoscope system according to the first embodiment. FIG. 3 is a diagram explaining a distal end configuration of an endoscope according to the first embodiment.

An endoscope system 1 illustrated in FIG. 1 and FIG. 2 includes an endoscope 2 that captures an in-vivo image of a subject by inserting a distal end portion into a body of the subject, a light source device 3 that generates illumination light emitting from a distal end of the endoscope 2, a processing device 4 that performs predetermined signal processing on an imaging signal captured by the endoscope 2, and that comprehensively controls operation of the entire endoscope system 1, a display device 5 that displays the in-vivo image generated by the signal processing of the processing device 4, and a treatment device 6.

The endoscope 2 includes an insertion portion 21 that has a flexible thin long shape, an operating portion 22 that is connected to a proximal end side of the insertion portion 21, and that accepts an input of various kinds of operation signals, and a universal cord 23 that extends in a direction different from a direction in which the insertion portion 21 extends from the operating portion 22, and that has various kinds of cables connected to the light source device 3 and the processing device 4 therein.

The insertion portion 21 includes a distal end portion 24 having an imaging device 244 in which pixels that generates a signal by receiving light and performing photoelectric conversion are arranged in a two-dimensional array, a bendable portion 25 that is constituted of multiple bending elements and is bendable, and a flexible tube portion 26 that is connected to a proximal end side of the bendable portion 25, and that has a flexible long shape. The insertion portion 21 is inserted into a body cavity of a subject, and captures images of an object, such as a living tissue located in a position at which external light cannot reach, by using the imaging device 244.

The operating portion 22 includes a bending knob 221 that bends the bendable portion 25 in an up and down directions and a left and right directions, a treatment-tool insertion portion 222 that inserts treatment tools, such as a therapeutic light irradiation device, a biopsy forceps, an electrosurgical knife, and an examination probe, into a body cavity of the subject, and multiple switches 223 that are operation input portions to input an operation instruction signal of a peripheral device, such as an air feeder unit, a water feeder unit, and a screen display control, in addition to the processing device 4. The treatment tool inserted from the treatment-tool insertion portion 222 protrudes out from an opening portion through a treatment tool channel (not illustrated) of the distal end portion 24 (refer to FIG. 3).

The universal cord 23 has at least a light guide 241 and a bundle cable 245 including one or more signal lines thereinside. The universal cord 23 branches at an end portion on the opposite side to a side connected to the operating portion 22. At the branched end portion, a connector 231 that is detachable to the light source device 3 and a connector 232 that is detachable to the processing device 4 are provided. In the connector 231, a portion of the light guide 241 extends out from its end. The universal cord 23 propagates illumination light emitted from the light source device 3 to the distal end portion 24 through the connector 231 (the light guide 241), the operating portion 22, and the flexible tube portion 26. Moreover, the universal cord 23 transmits an image signal captured by the imaging device 244 arranged at the distal end portion 24 to the processing device 4 through the connector 232. The bundle cable 245 includes a signal line to transmit an imaging signal, a signal line to transmit a driving signal to drive the imaging device 244, and a signal line to transmit and receive information including unique information relating to the endoscope 2 (the imaging device 244). In the present embodiment, it is explained supposing that an electrical signal is transmitted using the signal line, but it may be configured to transmit an optical signal, or it may be configured to transmit a signal between the endoscope 2 and the processing device 4 by wireless communications.

The distal end portion 24 includes the light guide 241 that is constituted of a glass fiber or the like, and that forms a light guide for light emitted by the light source device 3, an illumination lens 242 that is arranged at a distal end of the light guide 241, an optical system 243 for light collection, and the imaging device 244 that is arranged at an image forming position of the optical system 243, and that receives light collected by the optical system 243, photoelectric-converts into an electrical signal, and performs predetermined signal processing.

The optical system 243 is constituted of one or more lenses. The optical system 243 forms an observed image on a light-receiving surface of the imaging device 244. The optical system 243 may have an optical zoom function to change an angle of view and a focus function to adjust a focus.

The imaging device 244 generates an electrical signal (image signal) by subjecting light from the optical system 243 to photoelectric conversion. The imaging device 244 is constituted of multiple pixels, each of which has a photodiode accumulating an electric charge according to light intensity, and a capacitor converting the electric charge transferred from the photodiodes into a voltage level, arranged in a matrix configuration. In the imaging device 244, each of the pixels generates an electrical signal by performing photoelectric conversion on light entering through the optical system 243, and electrical signals generated by pixels that are arbitrarily set as the readout targets among the pixels are sequentially read out, to be output as image signals. The imaging device 244 is implemented, for example, by using a charge coupled device (CCD) image sensor, or a complementary metal oxide semiconductor (CMOS) image sensor.

The endoscope 2 has a memory (not illustrated) that stores an execution program for the imaging device 244 to execute respective actions, a control program, and data including identification information of the endoscope 2. The identification information includes unique information (ID) of the endoscope 2, year of manufacture, specification information, and transmission method, and the like. Moreover, the memory may temporarily store image data generated by the imaging device 244.

A configuration of the light source device will be explained. The light source device 3 includes a light source unit 31, an illumination control unit 32, and a light source driver 33. The light source unit 31 switches illumination lights of various exposure levels to emit to a subject (specimen).

The light source unit 31 is constituted of one or more lenses, and the like, and emits light (illumination light) by driving a light source. Light generated by the light source unit 31 is emitted to the subject from a distal end of the distal end portion 24 through the light guide 241. The light source unit 31 has a white light source 311.

The white light source 311 emits light (white light) having a wide wavelength band in a visible range. The white light source 311 is implemented by using a light source of either one of a laser light source, a xenon lamp, a halogen lamp, and the like other than an LED light source.

The illumination control unit 32 controls an amount of power to be supplied to the light source unit 31, a light source to emit light, and driving timing of the light source based on a control signal (light control signal) from the processing device 4.

The light source driver 33 causes the light source unit 31 to emit light by supplying an electric current to a light source subject to emission of light under a control of the illumination control unit 32.

A configuration of the processing device 4 will be explained. The processing device 4 includes an image processing unit 41, a synchronization-signal generating unit 42, an input unit 43, a control unit 44, and a storage unit 45.

The image processing unit 41 receives image data of illumination light of respective colors captured by the imaging device 244 from the endoscope 2. When analog image data is received from the endoscope 2, the image processing unit 41 performs A/D conversion to generate a digital imaging signal. Moreover, when image data is received as an optical signal from the endoscope 2, the image processing unit 41 performs photoelectric conversion to generate digital image data.

The image processing unit 41 generates an image by performing predetermined image processing with respect to image data received from the endoscope 2, to output it to the display device 5, sets an enhanced region determined based on the image, and calculates a temporal variation of fluorescence intensity. The image processing unit 41 includes a white-light-image generating unit 411, a fluorescence-image generating unit 412, a tone-range setting unit 413, and a tone-expanded-image generating unit 414.

The white-light-image generating unit 411 generates a white light image based on an image formed with white light.

The fluorescence-image generating unit 412 generates a fluorescence image based on an image formed with fluorescence.

The tone-range setting unit 413 sets a range (for example, a range of brightness, and the like) of an image in which tone setting is performed based on the fluorescence intensity.

The tone-expanded-image generating unit 414 generates a tone-expanded image by allocating tones based on the tone range set by the tone-range setting unit 413. The tone-expanded-image generating unit 414 generates a tone-expanded image in which, for example, the tone of a portion of a fluorescence image is enhanced.

The white-light-image generating unit 411, the fluorescence-image generating unit 412, and the tone-expanded-image generating unit 414 generate an image by performing predetermined image processing. The predetermined image processing includes synchronization processing, tone correction processing, and color correction processing. The synchronization processing is processing to synchronize image data of respective color components of RGB. The tone correction processing is processing to perform correction of tones with respect to image data. The color correction processing is processing to perform color correction with respect to image data. The white-light-image generating unit 411, the fluorescence-image generating unit 412, and the tone-expanded-image generating unit 414 may adjust a gain according to a brightness of the image.

The image processing unit 41 is composed of a general-purpose processor, such as a central processing unit (CPU), or a dedicated processor of various kinds of arithmetic circuits having a specific function, such as an application specific integrated circuit (ASIC). The image processing unit 41 may have a configuration including a frame memory that holds an R-image data, G-image data, and B-image data.

The synchronization-signal generating unit 42 generates a clock signal (synchronization signal) to be serves as the basis for operation of the processing device 4, and outputs the generated synchronization signal to the light source device 3, the image processing unit 41, the control unit 44, and the endoscope 2. The synchronization signal generated by the synchronization-signal generating unit 42 includes a horizontal synchronization signal and a vertical synchronization signal.

Therefore, the light source device 3, the image processing unit 41, the control unit 44, and the endoscope 2 operate in synchronization to one another based on the generated synchronization signal.

The input unit 43 is implemented by a keyboard, a mouse, a switch, and a touch panel, and accepts input of various kinds of signals, such as an operation instruction signal to instruct an operation of the endoscope system 1. The input unit 43 may include a switch arranged on the operating portion 22 and a portable terminal, such as an external tablet computer.

The control unit 44 performs drive control of the respective components including the imaging device 244 and the light source device 3, input/output control of information with respect to the respective components, and the like. The control unit 44 refers to control information data for imaging control (for example, readout timing and the like) that is stored in the storage unit 45, and transmits it to the imaging device 244 as a driving signal through a predetermined signal line included in the bundle cable 245. Moreover, the control unit 44 may switch modes according to light to be observed. The control unit 44 switches, for example, between a normal observation mode to observe an image acquired by illumination of white light, and a fluorescence observation mode to observe a fluorescence image acquired by illumination of therapeutic light. The control unit 44 is composed of a general-purpose processor, such as CPU, or a dedicated processor of various kinds of arithmetic circuits performing specific functions, such as ASIC.

The storage unit 45 stores various kinds of programs to operate the endoscope system 1, and data including various kinds of parameters that are necessary for operation of the endoscope system 1. Furthermore, the storage unit 45 stores identification information of the processing device 4. The identification information includes unique information (ID) of the processing device 4, year of manufacture, specification information, and the like.

Moreover, the storage unit 45 stores various kinds of programs including an image-acquisition processing program to perform an image-acquisition processing method of the processing device 4. The various kinds of programs can be recorded on computer-readable recording medium, such as a hard disk, a flash memory, a compact disk read-only memory (CD-ROM), a digital versatile disk read-only memory (DVD-ROM), and a flexible disk, to be distributed widely. The various kinds of programs described above can be acquired by downloading them through a communication network. The communication network herein is implemented by, for example, an existing public switched network, a local area network (LAN), a wide area network (WAN), and the like, and can be wired or wireless.

The storage unit 45 with the above configuration is implemented by using a ROM in which various kinds of programs and the like have been preinstalled, a random access memory (RAM) or a hard disk that stores arithmetic parameters, data, and the like of respective processing, and the like.

The display device 5 displays an image for display corresponding to an image signal received from the processing device 4 (the image processing unit 41) through a video cable. The display device 5 is constituted of a liquid crystal or an organic electro luminescence (EL) monitor or the like.

The treatment device 6 includes a treatment-tool operating unit 61, and a flexible treatment tool 62 that extends from the treatment-tool operating unit 61. The treatment tool 62 used for PIT is a therapeutic light emitting unit that emits light for treatment (hereinafter, “therapeutic light”). The treatment-tool operating unit 61 controls emission of therapeutic light of the treatment tool 62. The treatment-tool operating unit 61 includes an operation input unit 611. The operation input unit 611 is constituted of, for example, a switch and the like. The treatment-tool operating unit 61 causes the treatment tool 62 to emit therapeutic light in response to input to the operation input unit 611 (for example, depression of a switch). In the treatment device 6, a light source that emits the therapeutic light may be arranged in the treatment tool 62, or may be arranged in the treatment-tool operating unit 61. The light source is implemented by using a semiconductor laser, an LED, or the like. The therapeutic light is, for example, light having a wavelength band of 680 nm or higher in the case of PIT, and is, for example, light with a central wavelength of 690 nm.

An illumination optical system included in the treatment tool 62 may have a configuration in which an irradiation range of therapeutic light can be changed. For example, it is constituted of an optical system that can change a focal length, a digital micromirror device (DMD), or the like, and is capable of changing a spot diameter and a shape of an irradiation range under the control of the treatment-tool operating unit 61.

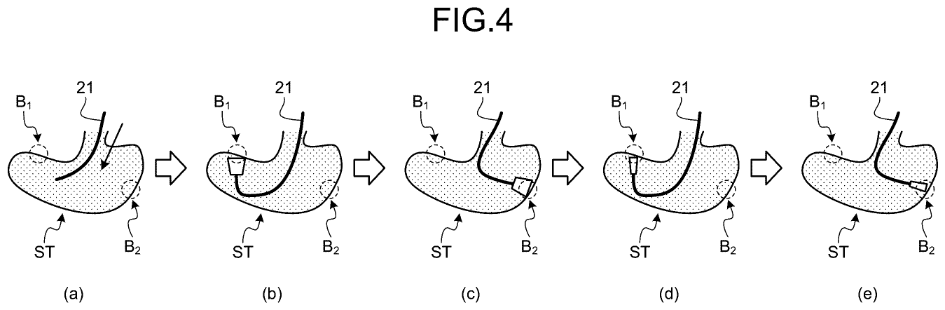

Subsequently, a flow of treatment using the endoscope 2 will be explained, referring to FIG. 4. FIG. 4 is a diagram illustrating an example of a flow of treatment using the endoscope according to the first embodiment of the disclosure. FIG. 4 is a diagram illustrating an example of execution of PIT, and treatment is performed by inserting the insertion portion 21 into a stomach ST.

First, an operator inserts the insertion portion 21 into the stomach ST (refer to (a) in FIG. 4). At this time, the operator causes the light source device 3 to irradiate white light, and searches for a treatment position while observing white light images inside the stomach ST displayed on the display device 5. In this example, as treatment targets, tumors B₁, B₂ are treated. In this process, administration of an antibody drug is performed for the tumors B₁ and B₂, which are treatment target portions. The administration of the antibody drug may be performed by using the endoscope 2, may be performed by using other devices, or may be performed by having a patient take the drug orally.

The operator determines a region including the tumors B₁ and B₂ as an irradiation area by observing the white light images. Moreover, excitation light or the like may be irradiated to the irradiation area as necessary.

The operator directs the distal end portion 24 toward the tumor B₁, and irradiates therapeutic light to the tumor B₁ by making the treatment tool 62 protrude from the distal end of the endoscope 2 (refer to (b) in FIG. 4). By the irradiation of therapeutic light, the antibody drug bound to the tumor B₁ reacts, and treatment is administered to the tumor B₁.

The operator then directs the distal end portion 24 toward tumor B₂, and irradiates therapeutic light to the tumor B₂ by making the treatment tool 62 protrude from the distal end of the endoscope 2 (refer to (c) in FIG. 4). To the irradiation of therapeutic light, the antibody drug bound to the tumor B₂ reacts, and treatment is applied to the tumor B₂.

Thereafter, the operator directs the distal end portion 24 toward tumor B₁, and irradiates therapeutic light and excitation light to the tumor B₁ from the distal end of the endoscope 2 (refer to (d) in FIG. 4). The operator checks a treatment effect on the tumor B₁ by acquiring a fluorescence image after the treatment. Confirmation of the treatment effect is determined by the operator by observing, for example, an image described later. In the present embodiment, an example of observing fluorescence acquired by the therapeutic light will be explained.

Moreover, the operator directs the distal end portion 24 toward tumor B₂, and irradiates therapeutic light to the tumor B₂ from the distal end of the endoscope 2 (refer to (e) in FIG. 4). The operator checks a treatment effect on the tumor B₂ by acquiring a fluorescence image after the treatment.

The operator repeats additional irradiation of therapeutic light and check of a treatment effect as necessary.

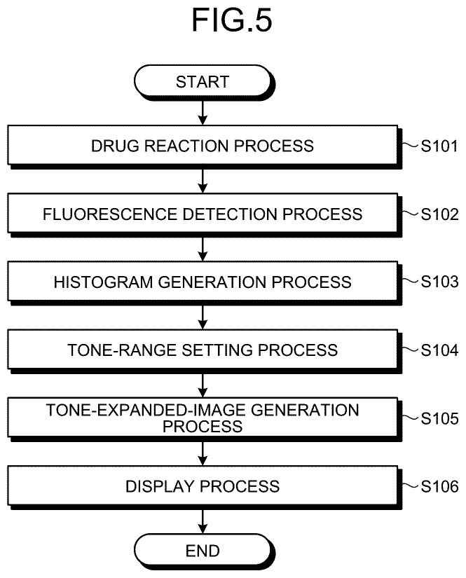

Subsequently, processing in the processing device 4 will be explained, referring to FIG. 5. FIG. 5 is a flowchart illustrating an example of processing of the processing device according to one embodiment of the disclosure. FIG. 5 illustrates processing in PIT when searching for a subject (in this example, a region including a cancer cell to which an antibody drug has been bound) by irradiating white light, irradiating therapeutic light to the subject, and confirming a treatment effect based on a fluorescence intensity. The operator searches for a treated position while observing a white light image displayed on the display device 5. The processing device 4 may change the observation mode according to light to be observed.

First, by the operation of the operator, therapeutic light is irradiated to the antibody drug bound to a cancer cell from the treatment tool 62, and the drug reacts (step S101: DRUG REACTION PROCESS). In this drug reaction process, a treatment in which the antibody drug is activated by irradiation of infrared ray, which is the therapeutic light, to destroy a cancer cell is performed.

The control unit 44 may set the observation mode to the fluorescence-light observation mode depending on irradiation of the therapeutic light. Determination of therapeutic light emission by the control unit 44 at this time is triggered by, for example, either one of reception of an operation signal by the operation input unit 611, or input of a therapeutic light irradiation start to the input unit 43 by the operator.

In a state in which the therapeutic light is being irradiated, the endoscope 2 detects fluorescence generated by the therapeutic light (step S102: FLUORESCENCE DETECTION PROCESS). By irradiation of the therapeutic light, the antibody drug of the subject is excited and emits fluorescence. The fluorescence-image generating unit 412 generates a fluorescence image or a white light image including fluorescence based on the imaging signal.

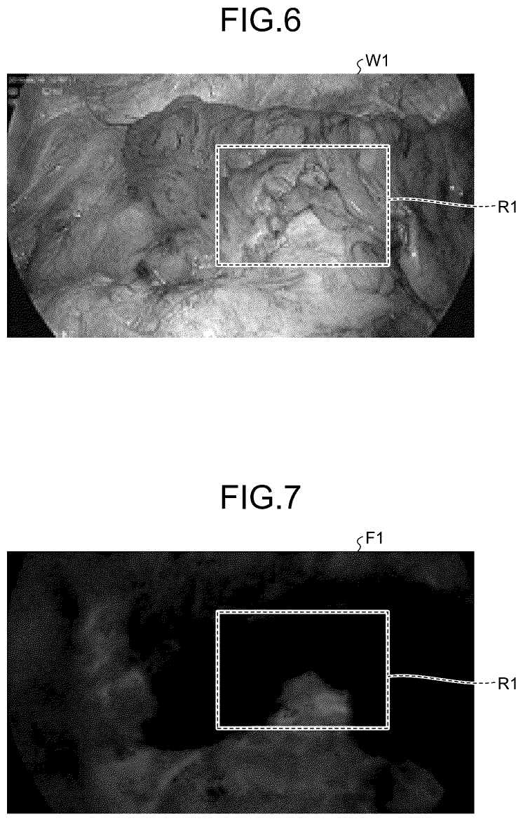

FIG. 6 is a diagram illustrating an example of an observation image including a fluorescence image. FIG. 7 is a diagram illustrating a fluorescence image that indicates the fluorescence intensity in the observation image illustrated in FIG. 6. FIG. 6 illustrates an example of an observation image that is acquired when white light and therapeutic light are irradiated. In an observation image W1 illustrated in FIG. 6, reflected light illuminated by white light is represented, and fluorescence emitted from the antibody drug upon irradiation of therapeutic light is represented. In a fluorescence image F1 illustrated in FIG. 7, an image formed by fluorescence is represented. The fluorescence image can be generated by separating only fluorescence by prism or the like in the imaging device 244 and by receiving white light and fluorescence at positions different from each other, or by selectively passing fluorescence through a filter or the like.

The observation image W1 and the fluorescence image F1 are images obtained by capturing the same area of the subject. Moreover, in the observation image W1 and the fluorescence image F1, a region of interest R1 is set. The region of interest may be preset by designating a position in an image, may be set arbitrarily by an operator by an input to the input unit 43, or may be set by the control unit 44 by detecting a feature portion of the image. Furthermore, the control unit 44 adjusts the position of the region of interest according to corresponding positions between images acquired at different times, or between the white light image and the fluorescence image, to match. At this time, the control unit 44 detects a corresponding position between images by a publicly-known method, such as pattern matching.

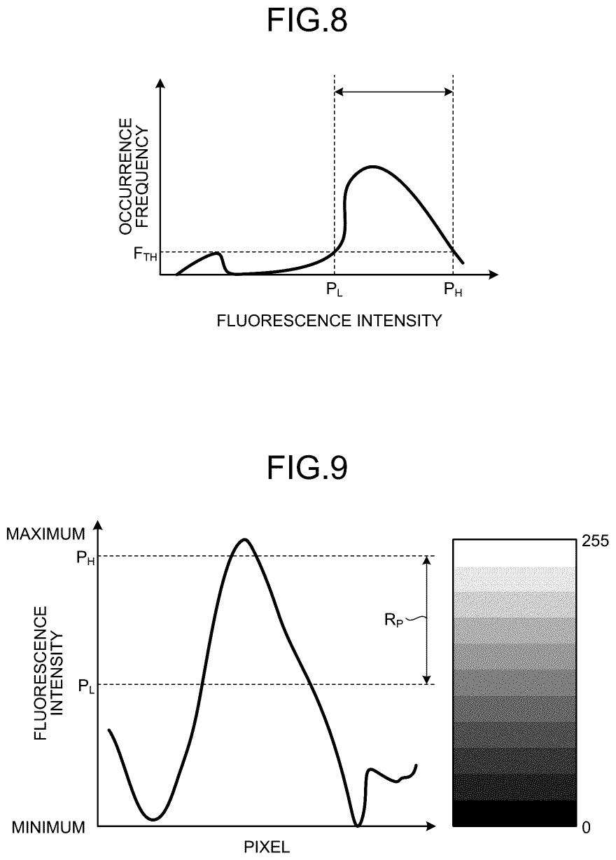

Thereafter, the tone-range setting unit 413 generates a histogram indicating occurrences frequency of fluorescence intensity (step S103: HISTOGRAM GENERATION PROCESS). FIG. 8 is a diagram illustrating a relationship between the fluorescence intensity and the occurrence frequency of in a region of interest. FIG. 8 is a histogram indicating the fluorescence intensity and the occurrence frequency. The tone-range setting unit 413 generates, for example, a histogram illustrated in FIG. 8. In this histogram, for the fluorescence intensity, brightness of a fluorescence image in a region of interest is used. The brightness may be divided into preset intervals, and may be divided into, for example, 10 ranges from 0 to 255. Specifically, for example, dividing into ranges of 0 to 25, 24 to 51, 52 to 76, 77 to 102, 103 to 127, 128 to 153, 154 to 178, 179 to 204, 205 to 229, and 230 to 255, and each pixel is allocated according to its brightness.

The tone-range setting unit 413 performs tone range setting after generation of a histogram (step S104: TONE-RANGE SETTING PROCESS). The tone-range setting unit 413 sets the fluorescence intensity equal to or higher than a preset threshold F_{TH} of occurrence frequency as a tone adjustment range. For example, in FIG. 8, a range between a fluorescence intensity P_{L} and a fluorescence intensity P_{H} is set as the tone adjustment range. Thereafter, the tone-range setting unit 413 sets a range of fluorescence intensity corresponding to the tone adjustment range in the fluorescence intensity distribution of each pixel of the fluorescence image to a tone range.

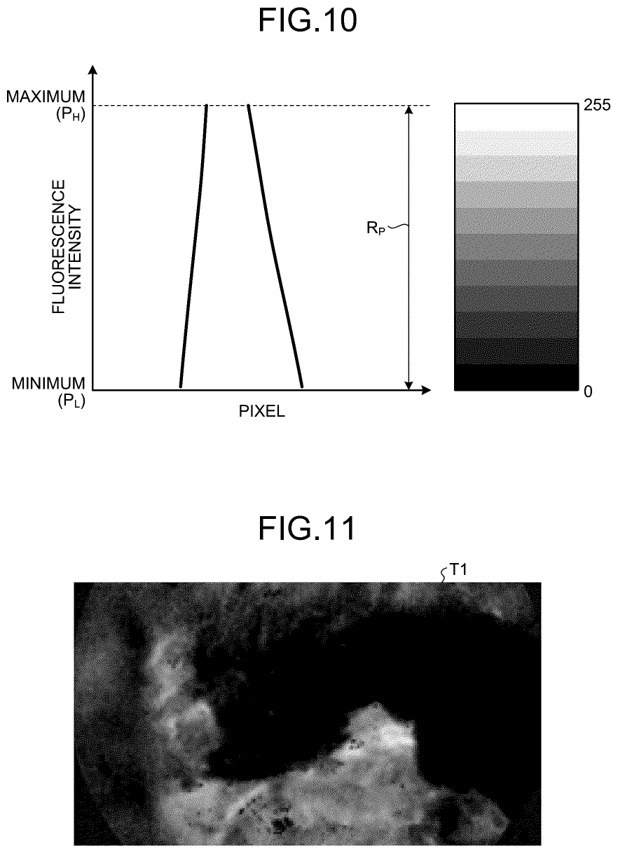

FIG. 9 and FIG. 10 are diagrams illustrating an example of the fluorescence intensity distribution with respect to pixels of a fluorescence image. In the fluorescence intensity distribution before tone adjustment illustrated in FIG. 9, for example, tones are set with respect to the minimum fluorescence intensity (for example, 0) to the maximum fluorescence intensity (for example, a upper limit value of brightness (255)). On the other hand, in the fluorescence intensity distribution after tone adjustment illustrated in FIG. 10, tones are set with respect to the minimum fluorescence intensity P_{L} to the maximum fluorescence intensity P_{H} set based on the histogram. In this case, the brightness of the minimum fluorescence intensity P_{L} is set to 0, and the brightness of the maximum fluorescence intensity P_{H} is set to 255, and brightnesses are allocated within the tone adjustment range R_{P}. The tone expansion ratio corresponds to a value obtained by dividing the maximum tone value (255 in this example) by the maximum brightness value of the fluorescence image before tone adjustment.

After the tone range setting, the tone-expanded-image generating unit 414 generates a tone-expanded image in which brightness is allocated to a fluorescence intensity based on the tone range set at step S104 (step S105).

Thereafter, the control unit 44 displays the generated tone-expanded image on the display device 5 (step S106: DISPLAY PROCESS). FIG. 11 is a diagram illustrating an example of a tone-expanded image obtained by performing the tone expansion processing on the fluorescence image illustrated in FIG. 7. A tone-expanded image T1 illustrated in FIG. 11 is an image in which fluorescence is more clearly expressed than that in the fluorescence image illustrated in FIG. 7.

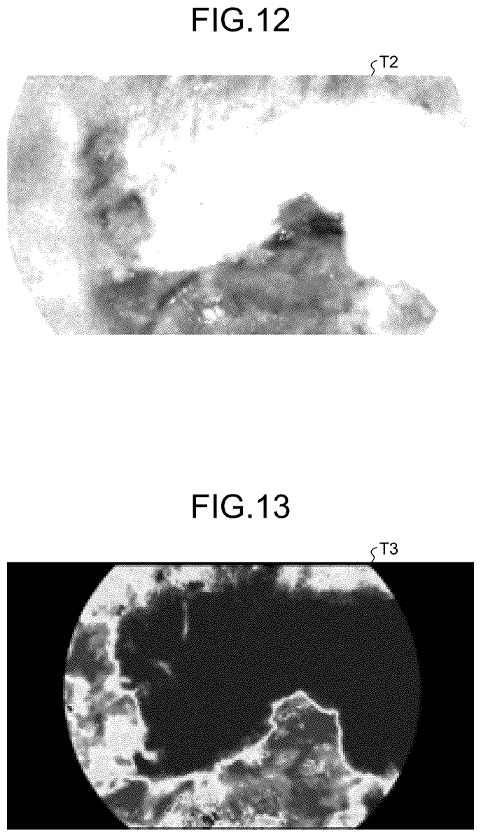

FIG. 12 and FIG. 13 are diagrams illustrating another display example of a tone-expanded image obtained by performing the tone expansion processing to the fluorescence image illustrated in FIG. 7. The tone-expanded-image generating unit 414 may generate a tone-expanded image T2 in which tones are inverted (refer to FIG. 12), or a tone-expanded image T3 representing a color map of fluorescence intensity in which colors according to fluorescence intensities are assigned (refer to FIG. 13), other than the tone-expanded image illustrated in FIG. 11.

In the first embodiment explained above, a range in which tones are adjusted is set based on a distribution of fluorescence intensity, and tones are allocated within a range between a minimum fluorescence intensity and a maximum fluorescence intensity in the set tone adjustment range, and a tone-expanded image expressing fluorescence clearly is thereby generated, to be displayed on the display device 5. According to the first embodiment, tones are set, limiting to a range occupied by majority of fluorescence intensity according to a distribution of fluorescence intensity of a fluorescence image, to generate a tone-expanded image and, therefore, changes in fluorescence intensity can be accurately grasped.

In PIT, using decrease in fluorescence intensity of a reagent during irradiation of therapeutic light, the progress of treatment is grasped based on the fluorescence intensity. In this case, when displaying a high initial fluorescence intensity to a low fluorescence intensity just before treatment completion using the same dynamic range, it becomes difficult to recognize partial intensity differences in a state in which the fluorescence intensity is low. Therefore, by selecting a range of fluorescence intensity to be displayed in response to the decrease in fluorescence intensity, by assigning a dynamic range to be displayed, and by expanding the tone in a region in which the fluorescence intensity is low, it becomes easy to recognize partial differences in fluorescence intensity in the region in which the treatment has progressed and the fluorescence intensity has decreased.

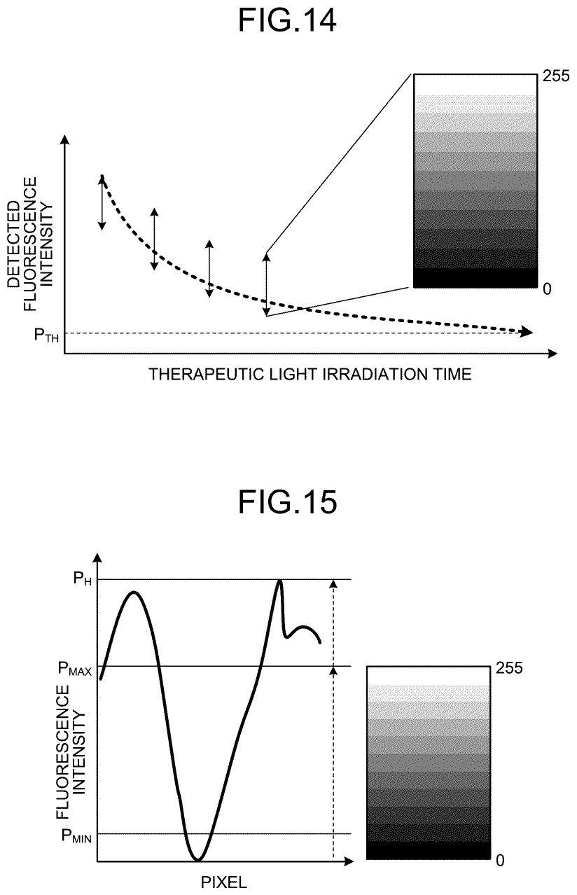

FIG. 14 is a diagram for explaining bout the tone expansion processing according to the first embodiment of the disclosure. By repeating the tone expansion processing according to fluorescence intensity, until it reaches a maximum attenuation value P_{TH}, and by setting tones, limiting to a range occupied by majority of fluorescence intensity according to a distribution of fluorescence intensity of a fluorescence image, changes in fluorescence intensity can be accurately grasped regardless of the magnitude of fluorescence intensity.

### Modification of First Embodiment

Next, a modification of the first embodiment will be explained. Because an endoscope system according to the modification is the same as the endoscope system 1 according to the first embodiment, explanation thereof will be omitted. In the modification, clipping processing to adjust a range of fluorescence intensity in which tone setting is performed according to a value of difference in fluorescence intensity is performed. FIG. 15 is a diagram for explaining about the clipping processing according to the modification of the first embodiment of the disclosure.

The tone-range setting unit 413 sets a range of fluorescence intensity in which the tone setting is performed according to a preset condition. In the present modification, a minimum value of the tone setting range is set to a preset minimum value P_{MIN}. Moreover, a maximum value of the tone setting range is set to a maximum value P_{MAX} obtained by multiplying the maximum value P_{H} of fluorescence intensity by a preset ratio. In the present modification, for example, 0.8 (80%) is set as the ratio. This ratio can be set arbitrarily. Moreover, the minimum value of the tone setting range can be also obtained by multiplying the minimum value of fluorescence intensity by a preset ratio.

According to the modification, an effect similar to that of the first embodiment can be obtained, and changes in fluorescence intensity in a portion of a region in which fluorescence is detected can be represented in detail.

### Second Embodiment

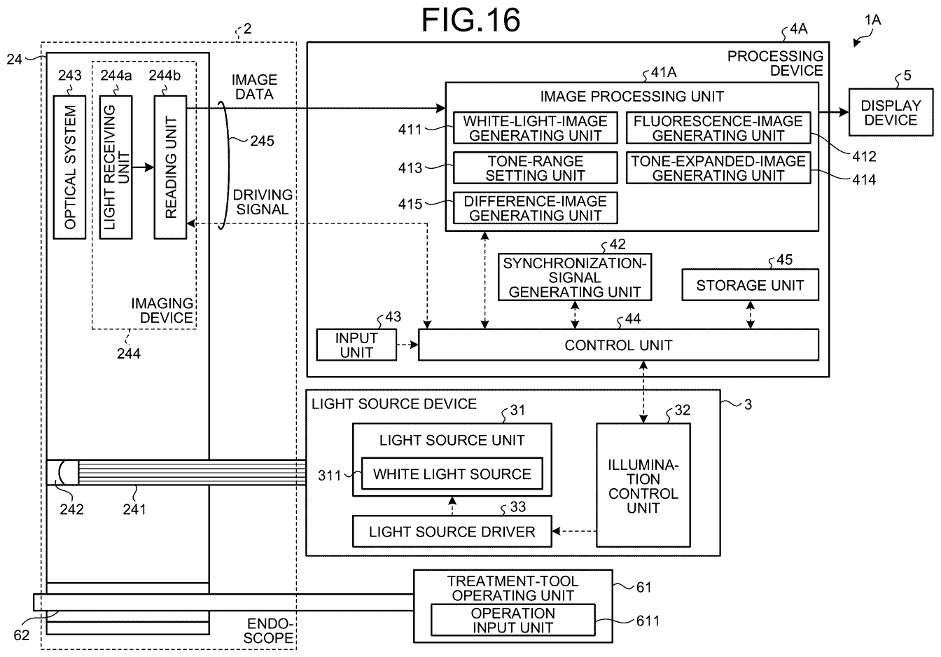

Next, a second embodiment will be explained. FIG. 16 is a block diagram illustrating a schematic configuration of an endoscope system according to the second embodiment of the disclosure. An endoscope system 1A according to the second embodiment includes a processing device 4A in place of the processing device 4 in the endoscope system 1 according to the first embodiment. A configuration other than the processing device 4A is the same as that of the endoscope system 1 and, therefore, explanation will be omitted.

A configuration of the processing device 4A will be explained. The processing device 4A includes an image processing unit 41A, the synchronization-signal generating unit 42, the input unit 43, the control unit 44, and the storage unit 45.

The image processing unit 41A includes the white-light-image generating unit 411, the fluorescence-image generating unit 412, the tone-range setting unit 413, the tone-expanded-image generating unit 414, and a difference-image generating unit 415.

The difference-image generating unit 415 generates a difference image that indicates a difference in fluorescence intensity in two fluorescence images acquired at times different from each other. The difference image is an image that takes a difference in fluorescence intensity between pixels that correspond to each other in position in the subject.

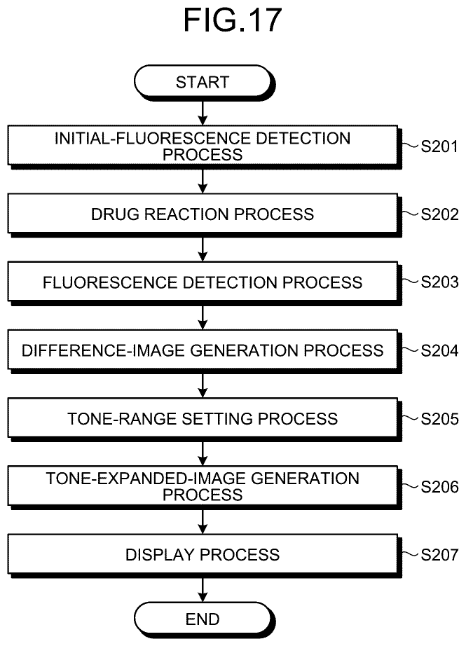

Subsequently, processing in the processing device 4A will be explained, referring to FIG. 17. FIG. 17 is a flowchart illustrating an example of processing of the processing device according to the second embodiment.

First, before starting treatment, therapeutic light is irradiated, to detect fluorescence before treatment (step S201: INITIAL-FLUORESCENCE DETECTION PROCESS). By this process, a fluorescence image before starting the treatment is acquired. This fluorescence image before starting the treatment is stored in the storage unit 45 as a reference fluorescence image.

Thereafter, by an operation of the operator, therapeutic light is irradiated to an antibody drug bound to a cancer cell from the treatment tool 62, and the drug reacts (step S202: DRUG REACTION PROCESS). By the drug reaction process, the therapeutic light is irradiated to the subject from the treatment tool 62, and treatment of destroying the cancer cell is performed.

The endoscope 2 detects fluorescence generated by the therapeutic light (step S203: FLUORESCENCE DETECTION PROCESS). By emission of the therapeutic light, the antidrug of the subject is excited to emit fluorescence. The fluorescence-image generating unit 412 generates a fluorescence image or a white light image including fluorescence based on an imaging signal.

Thereafter, the difference-image generating unit 415 generates a difference image between the fluorescence image before starting the treatment (reference fluorescence image) acquired at step S201 and the fluorescence image acquired at step S203 (step S204: DIFFERENCE-IMAGE GENERATION PROCESS).

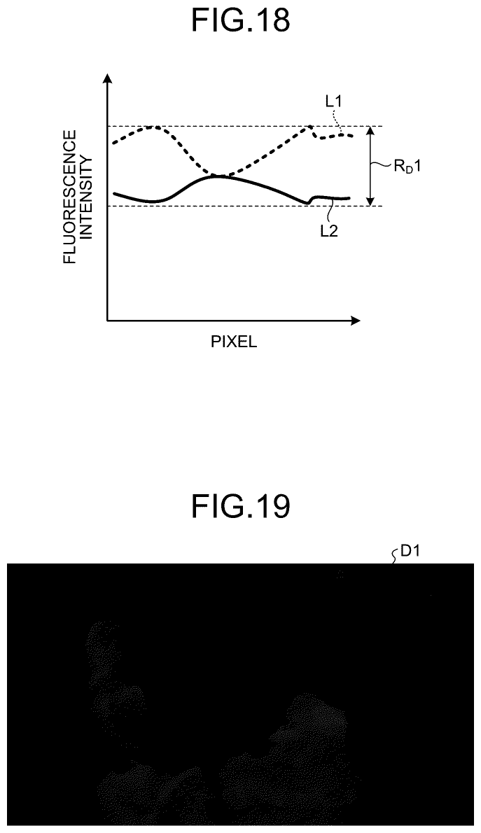

FIG. 18 is a diagram illustrating an example of the fluorescence intensity of corresponding pixels acquired at different times. A fluorescence intensity curve L1 illustrated in FIG. 18 indicates a fluorescence intensity of each pixel (for example, a pixel positioned at a region of interest) in the reference fluorescence image. Furthermore, a fluorescence intensity curve L2 indicates a fluorescence intensity of a pixel that is acquired at a different time from the reference fluorescence image, and that corresponds to the pixel position of the reference fluorescence intensity. The fluorescence intensity corresponds to a brightness value of the fluorescence image. In the fluorescence intensity curve L2 in FIG. 18, the fluorescence intensity in a portion in which reaction by the therapeutic light has progressed has decreased.

The difference-image generating unit 415 calculates a fluorescence intensity of the reference fluorescence image and a fluorescence intensity of the fluorescence image, and generates a difference image expressed by the difference. FIG. 19 is a diagram illustrating an example of the difference image. A difference image D1 illustrated in FIG. 19 is an image representing a difference (fluorescence change amount) between the fluorescence intensity of the reference fluorescence image and the fluorescence intensity of the fluorescence image. When the difference between the fluorescence intensity of the reference fluorescence image and the fluorescence intensity of the fluorescence image is small, it is difficult to visually recognize the difference. To represent the difference clearly, tones are set in a range corresponding to the maximum difference (maximum difference range). For example, in FIG. 18, a range R_{D}1 from the minimum value of the fluorescence difference (difference zero in this example) to the maximum value PD_{H} corresponds to the maximum difference range (also referred to as maximum difference range R_{D}1).

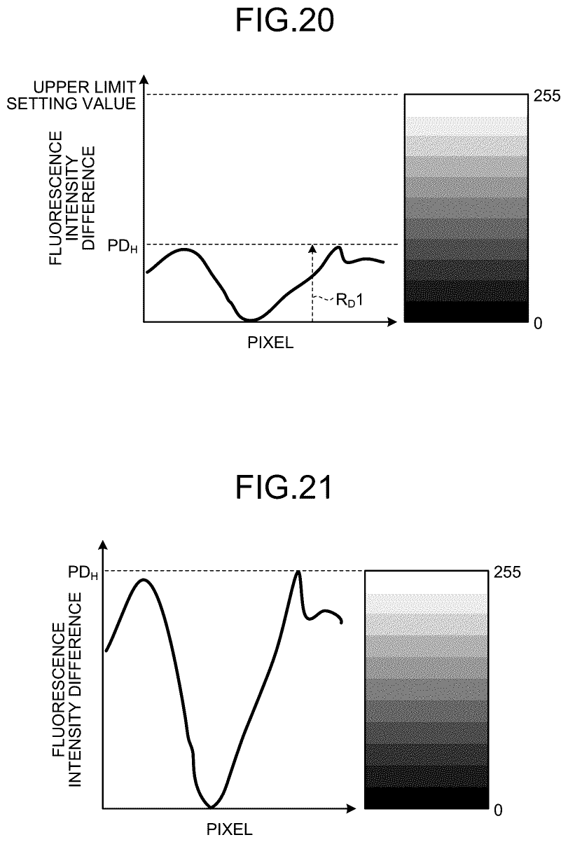

The tone-range setting unit 413 performs setting of a tone range after the difference image generation (step S205: TONE-RANGE SETTING PROCESS). FIG. 20 and FIG. 21 are diagrams illustrating an example of a distribution of fluorescence intensity difference with respect to a pixel of the difference image, and tone range. The tone-range setting unit 413 changes a tone range by changing a preset upper limit setting value of the fluorescence intensity difference, based on the maximum difference range R_{D}1. For example, the tone-range setting unit 413 sets the maximum difference range R_{D}1 to the tone adjustment range in FIG. 20. As the tone range is changed, brightness is allocated to the minimum value of the fluorescence difference to the maximum value D_{H} (refer to FIG. 21).

After the tone range setting, the tone-expanded image generating unit 414 generates a tone-expanded image in which brightness is allocated to the fluorescence intensity based on the tone range set at step S205 (step S206).

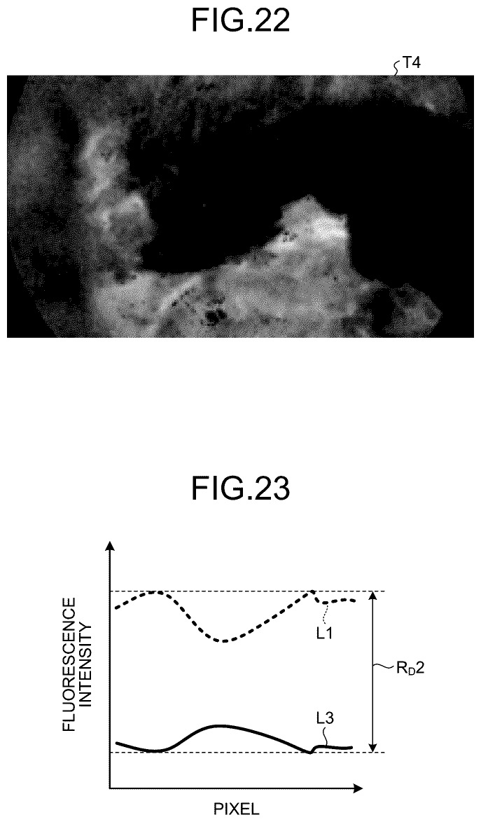

Thereafter, the control unit 44 displays the generated tone-expanded image on the display device 5 (step S207: DISPLAY PROCESS). FIG. 22 is a diagram illustrating an example of a tone-expanded image obtained by performing the tone expansion processing on the difference image illustrated in FIG. 19. A tone-expanded image T4 illustrated in FIG. 22 is an image in which the difference in fluorescence intensity is more clearly represented than the difference image illustrated in FIG. 19. Other than the tone-expanded image illustrated in FIG. 22, a tone-expanded image in which tones are inverted (refer to FIG. 12), or a tone-expanded image representing a color map of fluorescence intensity in which colors according to fluorescence intensities are assigned (refer to FIG. 13) may be generated.

Moreover, even when the fluorescence intensity has decreased sufficiently from the fluorescence intensity before treatment as the treatment has progressed, the tone adjustment processing described above is performed. FIG. 23 is a diagram illustrating an example of a fluorescence intensity of corresponding images acquired at different times. A fluorescence intensity curve L3 illustrated in FIG. 23 indicates a fluorescence intensity of a pixel of a fluorescence image that is acquired at a time when the treatment has progressed, which is a different time from the time at which the reference fluorescence image is acquired, and that corresponds to the pixel position of the reference fluorescence intensity.

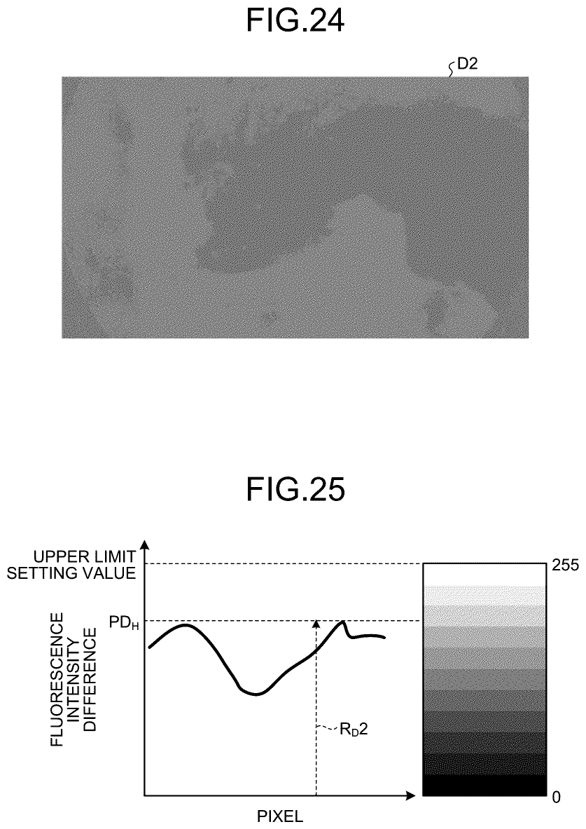

The difference-image generating unit 415 calculates a difference between the fluorescence intensity of the reference fluorescence image and the fluorescence intensity of the fluorescence image, and generates a difference image represented by the difference. FIG. 24 is a diagram illustrating an example of a difference image. A difference image D2 illustrated in FIG. 24 is an image representing a difference (florescence change amount) between the fluorescence intensity of the reference fluorescence and the fluorescence intensity of the fluorescence image.

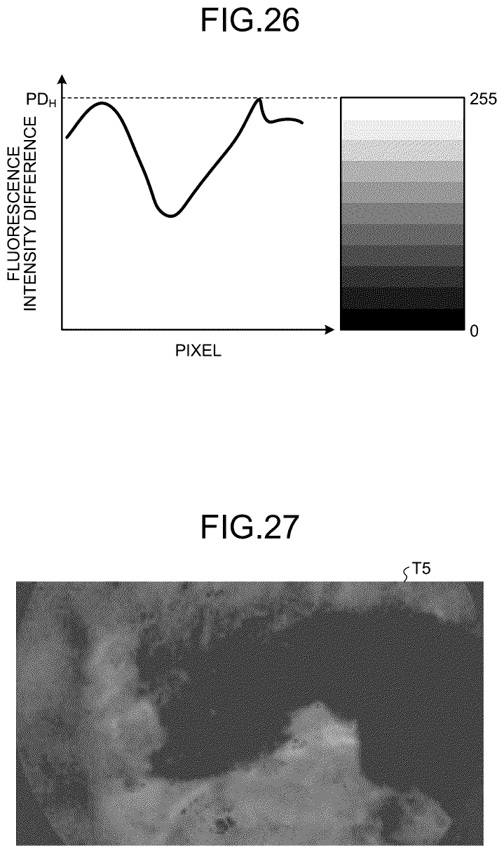

FIG. 25 and FIG. 26 are diagrams illustrating an example of a distribution of fluorescence intensity difference with respect to a pixel in the difference image, and a tone range. The tone-range setting unit 413 changes the tone range by changing a preset upper limit setting value of the fluorescence intensity difference, based on the maximum difference range R_{D}2. For example, the tone-range setting unit 413 sets the maximum difference range R_{D}2 to the tone adjustment range in FIG. 23. As the tone range is changed, brightness is allocated to the minimum value of the fluorescence intensity difference (zero in this example) to the maximum value PD_{H} (refer to FIG. 26).

After the tone range setting, the tone-expanded-image generating unit 414 generates a tone-expanded image in which brightness is allocated to the fluorescence intensity based on the set tone range. Thereafter, the control unit 44 displays the generated tone-expanded image on the display device 5. FIG. 27 is a diagram illustrating an example of a tone-expanded image obtained by performing the tone expansion processing on the difference image illustrated in FIG. 24. A tone-expanded image T5 illustrated in FIG. 27 is an image in which fluorescence is more clearly expressed than that in the difference image illustrated in FIG. 24.

In the second embodiment explained above, a range in which tones are adjusted is set based on a difference of fluorescence intensity, and tones are allocated in the set tone adjustment range, and a tone-expanded image expressing fluorescence clearly is thereby generated, to be displayed on the display device 5. According to the second embodiment, tones are set, limiting to a range occupied by majority of fluorescence intensity according to a distribution of fluorescence intensity of a fluorescence image, to generate a tone-expanded image and, therefore, changes in fluorescence intensity can be accurately grasped.

Moreover, in the second embodiment, a tone range is set with respect to a difference in fluorescence intensity, and tones of a difference image representing the difference are changed to be displayed and, therefore, an operator can visually recognize the change in the fluorescence intensity directly. According to the second embodiment, it is possible to let an operator grasp changes in fluorescence intensity further accurately.

In the second embodiment, the clipping processing of the modification described above can be adopted. In this case, the tone setting range is determined by a preset minimum value and a preset maximum value, or by the ratio to the difference.

### Third Embodiment

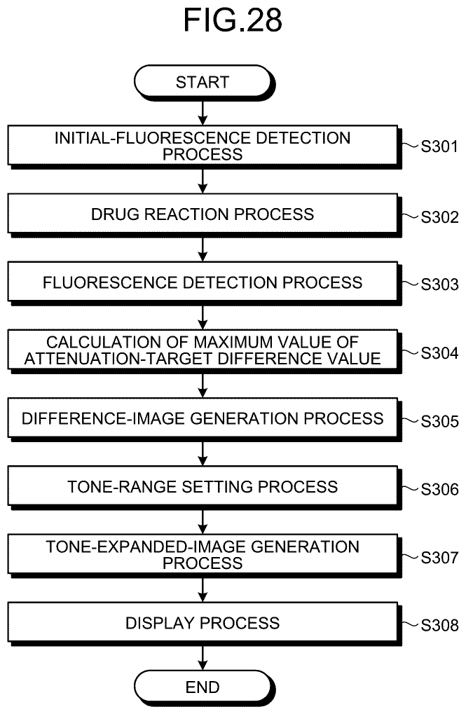

Next, a third embodiment will be explained. Because an endoscope system according to the third embodiment is the same as the endoscope system 1A according to the second embodiment, explanation thereof will be omitted. In the third embodiment, the storage unit 45 stores an attenuation target image of fluorescence intensity. This attenuation target image corresponds to a fluorescence image by which completion of treatment is determined. FIG. 28 is a flowchart illustrating an example of processing of a processing device according to the third embodiment.

First, before starting treatment, similarly to step S201 in FIG. 17, therapeutic light is irradiated, and fluorescence before treatment is detected (step S301: INITIAL-FLUORESCENCE DETECTION PROCESS).

Thereafter, by an operation of the operator, therapeutic light is irradiated to an antibody drug bound to a cancer cell from the treatment tool 62, and the drug reacts (step S302: DRUG REACTION PROCESS). By the drug reaction process, treatment of destroying the cancer cell is performed.

The endoscope 2 detects fluorescence generated by the therapeutic light (step S303: FLUORESCENCE DETECTION PROCESS). By emission of the therapeutic light, the antidrug of the subject is excited to emit fluorescence. The fluorescence-image generating unit 412 generates a fluorescence image or a white light image including fluorescence based on an imaging signal.

Thereafter, the control unit 44 refers to the storage unit 45 and reads out an attenuation target image, and calculates a maximum value of an attenuation target difference value between the attenuation target image and the reference fluorescence image (step S304: ATTENUATION-TARGET-VALUE CALCULATION PROCESS).

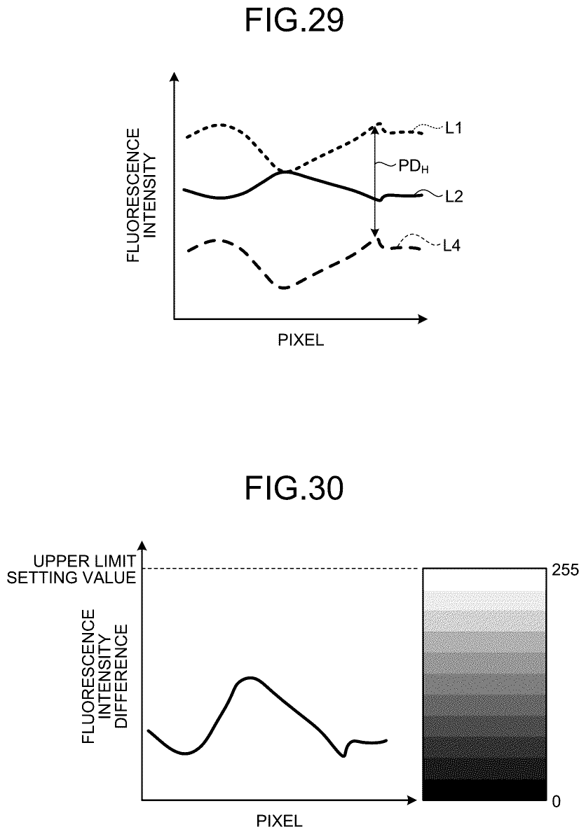

FIG. 29 is a diagram illustrating an example of fluorescence intensities of corresponding pixels acquired at different times. The fluorescence intensity curve L1 illustrated in FIG. 29 indicates a fluorescence intensity of each pixel (for example, pixel positioned in a region of interest) in the reference fluorescence image. Moreover, the fluorescence intensity curve L2 indicates a fluorescence intensity of a pixel of the fluorescence image acquired at a time different from a time of the reference fluorescence image, and that corresponds to the pixel position of the reference fluorescence intensity during the treatment. A fluorescence intensity curve L4 indicates a fluorescence intensity in the attenuation target image. A difference between the fluorescence intensity curve L1 and the fluorescence intensity curve L4 is to be the attenuation target difference value, and its maximum value is to be the maximum value PD_{H} of the attenuation difference.

Thereafter, the difference-image generating unit 415 generates a difference image between the fluorescence image before starting treatment (reference fluorescence image) acquired at step S201 and the fluorescence image acquired at step S203 (step S305: DIFFERENCE-IMAGE GENERATION PROCESS).

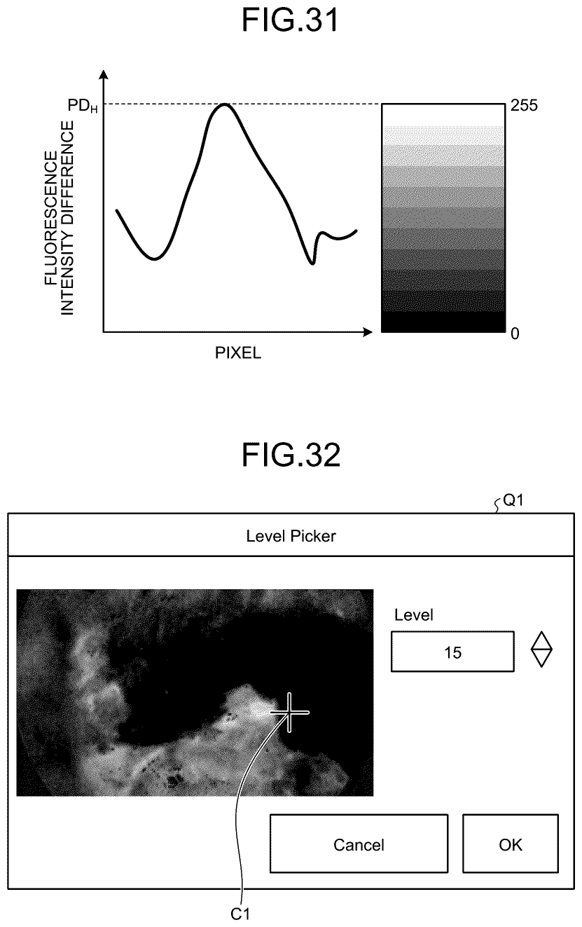

The tone-range setting unit 413 sets a tone range after the difference image generation (step S306: TONE-RANGE SETTING PROCESS). FIG. 30 and FIG. 31 are diagrams illustrating an example of a distribution of fluorescence intensity difference with respect to a pixel of a fluorescence image, and a tone range. The tone-range setting unit 413 changes a preset upper limit setting value (refer to FIG. 30) of the fluorescence intensity difference, based on the maximum difference range R_{D}1, to change the tone range. For example, in the case of a fluorescence image indicating the fluorescence intensity curve L2, the tone-range setting unit 413 calculates a difference between the fluorescence intensity curve L1 and the fluorescence intensity curve L4, and sets a range in which a maximum value is set to the maximum value PD_{H} to the tone adjustment range (refer to FIG. 31). As the tone range is changed, brightness is allocated from the minimum value (zero in this example) to the maximum value PD_{H} of the fluorescence intensity difference.

After the tone range setting, the tone-expanded image generating unit 414 generates a tone-expanded image in which brightness is allocated to the fluorescence intensity based on the tone range set at step S306 (step S307).

Thereafter, the control unit 44 displays the generated tone-expanded image on the display device 5 (step S308: DISPLAY PROCESS).

In the third embodiment explained above, a range in which tones are adjusted is set based on a difference of fluorescence intensity, and tones are allocated in the set tone adjustment range, and a tone-expanded image expressing fluorescence clearly is thereby generated, to be displayed on the display device 5. According to the third embodiment, tones are set, limiting to a range occupied by majority of fluorescence intensity according to a distribution of fluorescence intensity of a fluorescence image, to generate a tone-expanded image and, therefore, changes in fluorescence intensity can be accurately grasped.

Moreover, in the third embodiment, a tone range is set using the fluorescence intensity of the attenuation target, and a difference image representing the difference is displayed with changed tones and, therefore, changed in fluorescence intensity to determine completion of treatment can be confirmed further reliably. According to the third embodiment, it is possible to let an operator grasp changes in fluorescence intensity further accurately.

In the third embodiment, an example in which a tone range is set with the minimum value of fluorescence intensity set to zero in the tone setting processing has been explained, but an operator may set a minimum value with respect to a difference image and a fluorescence image. FIG. 32 is a diagram explaining about an example of setting a minimum value of a tone range. On a setting screen Q1 illustrated in FIG. 32, a level of fluorescence intensity (or a brightness value) at a position of a cursor C1 is displayed. The operator can place the cursor C1 at a position to be set as the minimum value, confirms a level of fluorescence intensity (or a brightness value) at the position, and set the level as the minimum value. This minimum value setting can be applied to the first and the second embodiments.

Furthermore, in the embodiment described above, an example in which treatment by therapeutic light and excitation of an antibody drug are performed has been explained, but it may be configured to irradiate excitation light to excite the antibody drug separately. In this case, for example, the light source device is configured to have an excitation light source that emits excitation light.

In the embodiment described above, an example in which the light source device 3 and the processing device 4 are separate units has been explained, but the light source device 3 and the processing device 4 may be configured to be an integrated. Moreover, in the embodiments, an example in which therapeutic light is irradiated by a treatment tool has been explained, but it may be configured such that the light source device 3 emits the therapeutic light.

Furthermore, in the embodiments described above, the endoscope system according to the disclosure has been explained as the endoscope system 1 using the flexible endoscope 2, an observation object of which is a living tissue or the like in a body of a subject, but it is applicable also to an endoscope system using a rigid endoscope, an industrial endoscope used to observe material properties, a fiberscope, and an optical endoscope, such as an optical scope, with a camera head attached to its eyepiece part.

As described above, the image processing apparatus, the photoimmunotherapy treatment system, the image processing method, and the image processing program are useful for accurately grasping changes in fluorescence intensity.

According to the disclosure, an effect of accurately grasping changes in fluorescence intensity is produced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus that processes a fluorescence image signal, the apparatus comprising a processor comprising hardware, the processor being configured to:
acquire a fluorescence image obtained by therapeutic light that causes a drug to react;
set a fluorescence intensity signal in which an occurrence frequency of a fluorescence intensity of the fluorescence image is equal to or higher than a threshold to a tone adjustment range of an image; and
allocate tones according to the set tone adjustment range to generate a tone-expanded image.

2. The image processing apparatus according to claim 1, wherein
the processor is configured to perform a clipping processing to set a tone range according to a condition preset with respect to any one of a maximum value and a minimum value of the fluorescence intensity signal in which the occurrence of fluorescence intensity of the fluorescence image is equal to or higher than the threshold.

3. The image processing apparatus according to claim 2, wherein
the processor is configured to set a tone range according to a ratio preset with respect to any one of the maximum value and the minimum value of the fluorescence intensity signal in which the occurrence of fluorescence intensity of the fluorescence image is equal to or higher than the threshold.

4. The image processing apparatus according to claim 1, wherein
the processor is configured to set a region of interest where a tone range is set with respect to the fluorescence image.

5. A fluorescence-image processing method, comprising:
acquiring, by a processor, a fluorescence image obtained by therapeutic light that causes a drug to react;
setting, by the processor, a fluorescence intensity signal in which an occurrence frequency of a fluorescence intensity of the fluorescence image is equal to or higher than a threshold to a tone adjustment range of an image; and
allocating, by the processor, tones according to the set tone adjustment range to generate a tone-expanded image.

6. A non-transitory computer-readable recording medium with an executable image processing program stored thereon, the program causing a computer to execute:
acquiring a fluorescence image obtained by therapeutic light that causes a drug to react;
setting a fluorescence intensity signal in which an occurrence frequency of a fluorescence intensity of the fluorescence image is equal to or higher than a threshold to a tone adjustment range of an image; and
allocating tones according to the tone adjustment range set at the setting to generate a tone-expanded image.

7. An image processing apparatus that processes a fluorescence image signal, the apparatus comprising a processor comprising hardware, the processor being configured to:
acquire an initial fluorescence image at a time of starting irradiation of therapeutic light that causes a drug to react;
acquire a fluorescence image during irradiation of the therapeutic light;
generate a difference image that represents a difference in a fluorescence intensity between the initial fluorescence image and the fluorescence image during irradiation of the therapeutic light;
set a tone range of an image with respect to the difference based on a distribution of a fluorescence intensity of the fluorescence image; and
allocate tones according to the set tone range to generate a tone-expanded image.

8. The image processing apparatus according to claim 7, wherein
the processor is configured to perform a clipping processing to set a tone range according to a condition preset with respect to any one of a maximum value and a minimum value of the difference in the difference image.

9. An image processing apparatus that processes a fluorescence image, the apparatus comprising a processor comprising hardware, the processor being configured to:
acquire a fluorescence image obtained by therapeutic light that causes a drug to react;
set a tone range of an image based on a distribution of a fluorescence intensity of the fluorescence image and on an attenuation target value set with respect to the fluorescence image; and
allocate tones according to the set tone range to generate a tone-expanded image.
